(19) United States
(12) Patent Application Publication
(10) Pub. No.: US 2026/0325117 A1
(43) Pub. Date: Oct. 22, 2026

(54) URINE COLLECTION FOR ANIMALS

(71) Applicant: QSM Diagnostics, Inc., Boston, MA (US)

(72) Inventor: Edgar D. GOLUCH, Somerville, MA (US)

(21) Appl. No.: 19/099,516

(22) PCT Filed: Aug. 1, 2023
(86) PCT No.: PCT/US2023/029191

§ 371 Date: Jan. 29, 2025

Related U.S. Application Data
(60) Provisional application No. 63/394,080, filed on Aug. 1, 2022.

(51) Int. Cl. A61B 10/00; A01K 23/00

(52) U.S. Cl. CPC A61B 10/007; A01K 23/005; A61B 2503/40

(57) ABSTRACT

The present disclosure relates to methods and devices for collection of urine.

### PRIORITY

The present application claims the benefit of and priority to U.S. Provisional Application No. 63/394,080, filed Aug. 1, 2022, the entire contents of which are hereby incorporated by reference.

### FIELD

The present disclosure relates to methods and devices for the collection of urine.

### BACKGROUND

Urine is routinely collected from animals for analysis to determine their health status and identify diseases. Urine may be collected in two ways, as a free catch or via a cystocentesis procedure. The collection of urine from small animals, particularly dogs, via either technique is challenging for multiple reasons. Cystocentesis is a clinical technique that obtains urine directly from the urinary bladder of the animal. Cystocentesis requires the insertion of a sterile needle, with a syringe attached, through the abdominal wall and bladder wall to obtain the urine sample. Guiding the needle into the bladder requires special training to not accidentally puncture a vein or another organ. The animal needs to be held in place during the procedure, the skin at the insertion site needs to be sterilized, and ideally a topical anesthetic is administered to minimize pain. The animal may not have urine in the bladder to collect and the procedure must be repeated after waiting for an hour or more. Conversely, the free catch technique has lower potential for complications, but can be challenging to execute. The free catch technique has a risk of sample contamination that may affect tests related to diagnosing infections. This risk is minimized by performing bacterial culture and sensitivity tests to distinguish contaminating microbes from infectious pathogens.

The free catch technique simply consists of placing a sterile cup beneath the urethra after the animal begins urinating. However, the animal is often startled by the presence of the cup, stops urinating, and begins moving around. Urine collected in the cup can be knocked out when the animal begins moving. Placement of the cup in the urine stream can also be challenging in small animals because there is little room around the legs and the urethra is low to the ground. Accordingly, there remains a need for novel methods of urine collection from animals.

### SUMMARY

Accordingly, the present disclosure provides, in part, a methods and devices for collecting urine that is smaller, less invasive, and reduces the chance of sample loss.

In aspects, the present disclosure provides a urine collection device comprising: (a) a urine collection substrate comprising a porous material, (b) a housing for the porous material that can detachably hold the porous material; and (c) a rod that is detachably attachable to the sponge housing.

In embodiments, the porous material is a sponge. In embodiments, the sponge is cellulose sponge or hydrophilic polyurethane sponge. In embodiments, the porous material is expandable upon contact with an aqueous liquid. In embodiments, the porous material is compressed and expands when it absorbs liquid. In embodiments, the porous material changes color when exposed to liquid.

In embodiments, the porous material does not substantially bind to one or more components of urine selected from proteins, electrolytes, creatinine, urine pigments (e.g., urochrome, and pigments from food such as beets), acids, bases, ammonia, glucose, ketones, blood cells, epithelial cells, bacteria, mucus, bilirubin, urinary casts, and urinary crystals. In embodiments, the porous material does not substantially release in urine sample absorbed in the porous material one of more of pigments, sugars, acids, bases, ketones, ammonia, bacteria and electrolytes. In embodiments, when a urine specimen that is extracted from the porous material that has absorbed a urine sample, the urine specimen does not substantially differ from the urine sample with respect to one or more of pH, specific gravity, and electrolytes.

In embodiments, the sponge has a capacity of absorbing at least 0.5 mL, or at least 1 mL, or at least 2 mL, or at least 5 mL, or at least 10 mL, or at least 20 mL aqueous liquid.

In embodiments, the rod is hingably attachable to the sponge housing. In embodiments, the rod is foldable and/or extendable. In embodiments, the rod is at least 6 inches, or at least 1 foot, or at least 2 feet long.

In embodiments, the urine collection device further comprises a handle.

In embodiments, the urine collection device: (a) holds the porous collection material in place, (b) minimizes the effort required by the collector to obtain the urine sample, and/or (c) minimizes the risk of sample contamination.

In aspects, the present disclosure provides a kit for urine collection comprising the urine collection device of any one of claims 1 to 14.

In embodiments, the kit further comprises one or more of a urine collection vial, a container to hold dry and/or wet porous material, a sterile air-tight container for transport, and instructions to use.

In aspects, the present disclosure provides a method for collecting urine, the method comprising: (a) obtaining a sample of urine by absorbing the urine into a porous material, (b) identifying when a sufficient volume of sample is collected, and (c) extracting the urine from the porous material.

In embodiments, the porous material is attached to a housing that detachably holds the porous material. In embodiments, the housing that detachably and hingably attached to a rod. In embodiments, the rod is at least 6 inches, or at least 1 foot, or at least 2 feet long.

In embodiments, the porous material is a sponge. In embodiments, the sponge material is cellulose. In embodiments, the porous material is compressed and expands when it absorbs liquid. In embodiments, the porous material changes color when exposed to liquid.

In embodiments, the porous material containing the sample is placed into a sterile air-tight container for transport.

In embodiments, the method further comprises extracting a liquid sample from the porous material.

In embodiments, the liquid sample is extracted from the porous material by compressing the porous material comprising the urine sample. In embodiments, the method further comprises transferring the liquid sample to a sterile air-tight container. In embodiments, the sample is removed from the sponge immediately before analysis at a laboratory.

In embodiments, the method: (a) minimizes the effort required by the collector to obtain the urine sample, and/or (b) minimizes the risk of sample contamination.

In aspects, the present disclosure provides a method for performing urine analysis, the method comprising collecting urine using the urine collection device of any one of the embodiments disclosed herein, or the kit of the embodiments disclosed herein, or the methos of any the embodiments disclosed herein and performing urine analysis.

In embodiments, the analysis is performed at a laboratory. In embodiments, the analysis comprises a quantitative analysis of urine protein and/or urine creatinine, urine dip stick analysis, urine sedimentation analysis, and/or urine culture identification.

### BRIEF DESCRIPTION OF THE DRAWINGS

FIG. 1A to FIG. 1F depict exemplary non-limiting instructions for urine sample collection. The sample collection is suitable for room temperature storage and shipping to a testing laboratory.

FIG. 2A to FIG. 2D depict photographs of the urine collection device before use.

FIG. 3 depicts that the sponge changes shape while collecting sample.

FIG. 4A and FIG. 4B depict a fully wetted sponge contains sufficient sample for analysis.

FIG. 5 depicts a sponge that is removed from the collection device so that it can be sent for analysis.

### DETAILED DESCRIPTION

Collecting urine sample for laboratory analysis is challenging for pets, companion animals, livestock, human babies, and some human patients.

Accordingly, the present disclosure provides, in part, a method and device for collecting urine that is smaller, less invasive, and reduces the chance of sample loss.

In aspects, the present disclosure provides a method for collecting urine. The method comprises: (a) obtaining a sample of urine by absorbing the urine into a porous material, (b) identifying when a sufficient volume of sample is collected, and (c) extracting the urine from the porous material. The porous material must not change the composition of the urine sample to allow for accurate measurement of the presence, absence, or amount of various compounds or microbes in the urine.

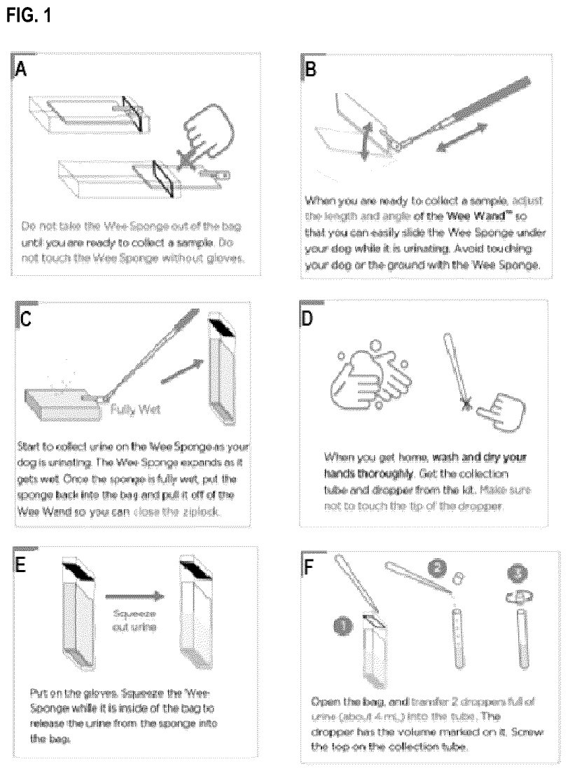

In embodiments, and in reference to FIGS. 1A-1F, an exemplary urine sample collection method using the present urine collection device is illustrated. In embodiments, the urine collection device is provided as part of a kit, as described herein. In embodiments, the urine samples are registered, for example via the Internet, email, or a web-based App, where a registration identifier, e.g., a numerical or alphanumerical code, is provided to more easily track and/or identify the sample(s). In embodiments, registration of one or more urine samples includes scheduling a pick-up or transportation arrangement, for example with one or more logistics companies that are suitable for transportation of urine samples.

In embodiments, and in reference to FIGS. 1A-1C, the porous material is protected in packaging material, for example in a sterile plastic bag, where the porous material is removed immediately prior to urine collection (e.g., as shown in FIG. 1A). In embodiments, the urine collection device includes a rod (e.g., “Wee Wand,” as indicated in FIG. 1B) hingably connected to the porous material (e.g., “Wee Sponge,” as indicated in FIG. 1B), where the length and angle of the urine collection device can be adjusted. In embodiments, the porous material expands and/or changes color in response to absorbing urine, where once wetted, the porous material (e.g., sponge) is removed from the urine collection device and sealed in a sterile container, such as a sealable plastic bag (e.g., as shown in FIG. 1C).

In embodiments, and in reference to FIGS. 1D-1F, the transfer of the urine from the porous material into one or more collection tubes is performed by the individual performing the urine collection, or performed by a secondary individual, e.g., a veterinarian or physician, laboratory technician, etc. In embodiments, the sealed porous material can be shipped for urine analysis. In embodiments, the wetted porous material (e.g., sponge) is expressed inside the sealed container to extract the absorbed urine (e.g., as shown in FIG. 1E). In embodiments, the urine collection kit includes one or more droppers used for extracting urine from the sealable container to aliquot the urine into one or more collection tubes (e.g., as shown in FIG. 1F).

In embodiments, the sample collection tubes are placed inside a second sealable transport container, e.g., a sealable biohazard bag, where the transport container includes absorbent material to prevent leakage of urine. In embodiments, the sample can be stored and/or shipped at room temperature. In embodiments, the urine collection kit includes a pre-paid shipping label to facilitate transport of the urine samples. In embodiments, the results are electronically transmitted, for example via the Internet, email, or a web-based App.

In embodiments, the porous material is a sponge. In embodiments, the sponge material is cellulose. In embodiments, the sponge is or comprises plastic, foamed polymer, polyester, polyurethane, and/or cellulose.

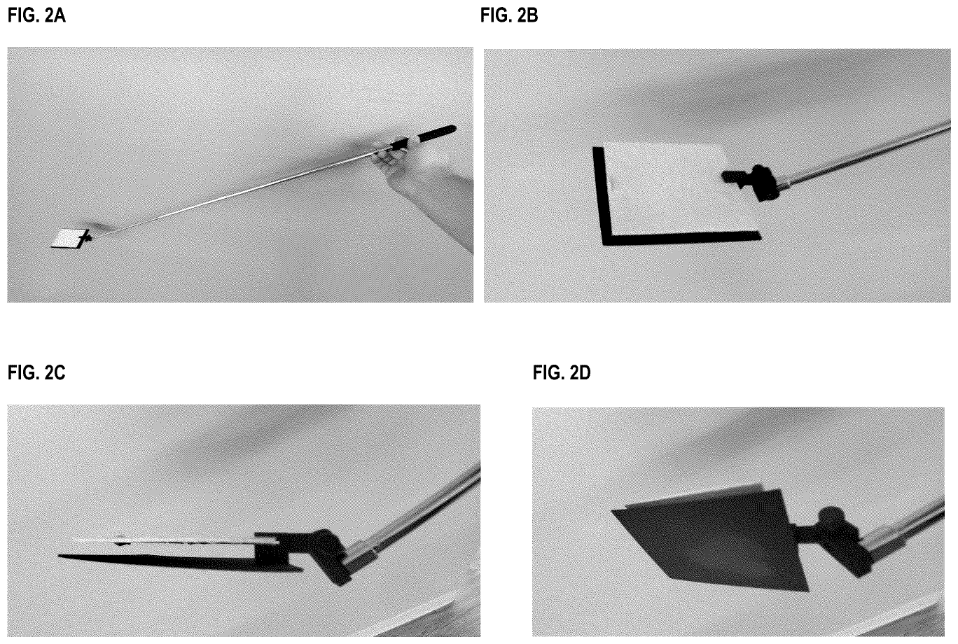

In embodiments, the porous material is attached to a device that minimizes the chances of contamination of the sample (e.g., as shown in FIGS. 2A-2D).

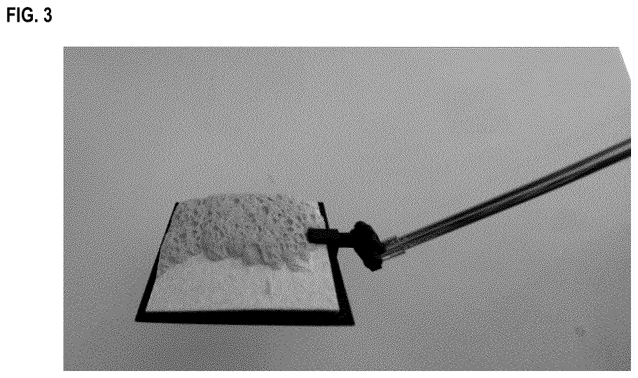

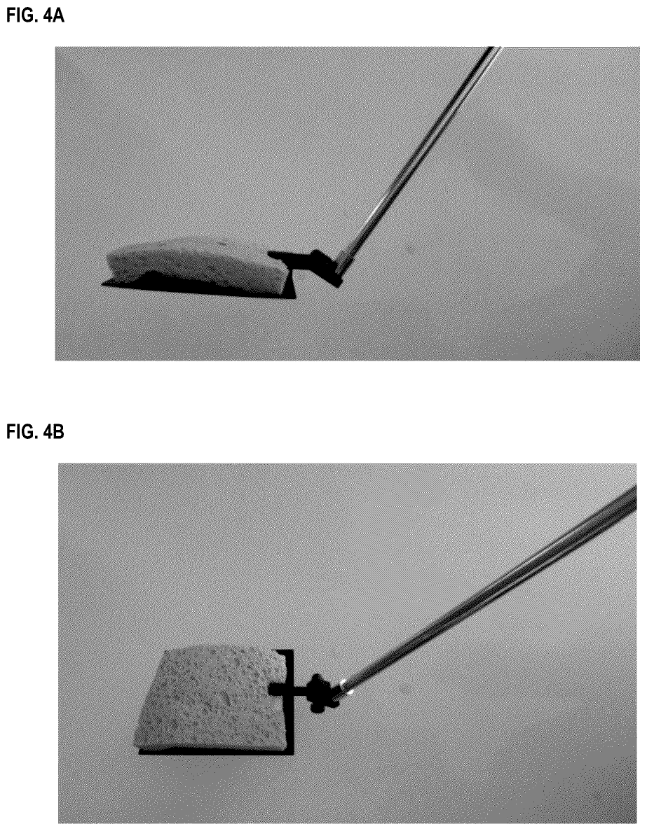

In embodiments, the porous material changes color when exposed to liquid (e.g., as shown in FIG. 3). In embodiments, the porous material is compressed and expands when it absorbs liquid (e.g., as shown in FIG. 4A-4B).

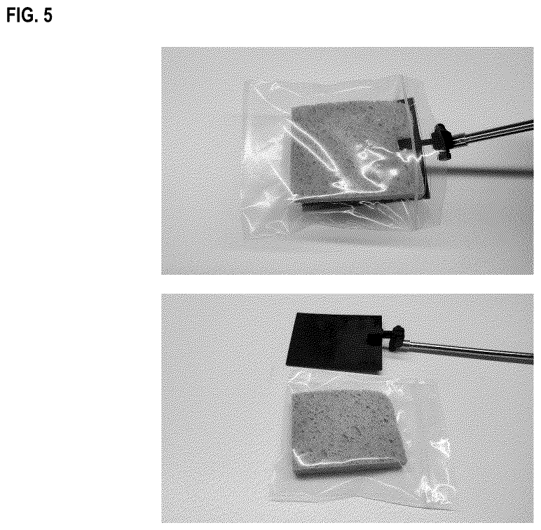

In embodiments, the porous material containing the sample is placed into a sterile, air-tight container for transport. In embodiments, the sterile, air-tight container is a sealable bag (e.g., as shown in FIG. 5).

In embodiments, the porous material containing the liquid sample is compressed, expressed, or otherwise exposed to pressure to release the sample.

In embodiments, the urine collection device: (a) holds the porous collection material in place, (b) minimizes the effort required by the collector to obtain the urine sample, and (c) minimizes the risk of sample contamination.

In embodiments, the collection device includes a support that prevents the sponge from contacting the ground.

In embodiments, the sample is removed from the sponge by the collecting individual.

In embodiments, the sample is removed from the sponge immediately before analysis at the laboratory.

In aspects, the present disclosure provides a urine collection device comprising: (a) a urine collection substrate comprising a porous material, (b) a housing for the porous material that can detachably hold the porous material; and (c) a rod that is detachably attachable to the sponge housing.

In embodiments, the porous material is a sponge. In embodiments, the sponge is cellulose sponge or hydrophilic polyurethane sponge. In embodiments, the porous material is expandable upon contact with an aqueous liquid. In embodiments, the porous material is compressed and expands when it absorbs liquid. In embodiments, the porous material changes color when exposed to liquid.

In embodiments, the porous material does not substantially bind to or otherwise alter the concentration of one or more components of urine selected from proteins, electrolytes, creatinine, urine pigments (e.g., urochrome, and pigments from food such as beets), acids, bases, ammonia, glucose, ketones, blood cells (e.g., white blood cells (WBCs), red blood cells (RBCs), epithelial cells, bacteria, mucus, bilirubin, urobilinogen, urinary casts, and urinary crystals (e.g., phosphate crystals, calcium oxide crystals, etc.). In embodiments, the porous material does not substantially alter the specific gravity of the urine. In embodiments, the porous material does not substantially release in urine sample absorbed in the porous material one of more of pigments, sugars, acids, bases, ketones, ammonia, bacteria and electrolytes. In embodiments, when a urine specimen that is extracted from the porous material that has absorbed a urine sample, the urine specimen does not substantially differ from the urine sample with respect to one or more of pH, specific gravity, and electrolytes.

In embodiments, the sponge has a capacity of absorbing at least 0.5 mL, or at least 1 mL, or at least 2 mL, or at least 5 mL, or at least 10 mL, or at least 20 mL aqueous liquid.

In embodiments, the rod is hingably attachable to the sponge housing. In embodiments, the rod is foldable and/or extendable. In embodiments, the rod is at least 6 inches, or at least 1 foot, or at least 2 feet long, or at least 3 feet long, or at least 4 feet long, or at least 5 feet long. In embodiments, the rod is composed of plastic, metal (e.g., aluminum, copper, iron, steel, etc.), wood, etc.

In embodiments, the urine collection device further comprises a handle. In embodiments, the handle includes a button, switch, or other means by which the sponge can be released from the rod, for example, by mechanically controlling the holder of the urine collection device holding the porous collection material in place.

In embodiments, the urine collection device: (a) holds the porous collection material in place, (b) minimizes the effort required by the collector to obtain the urine sample, and/or (c) minimizes the risk of sample contamination.

In aspects, the present disclosure provides a method for collecting urine, the method comprising: (a) obtaining a sample of urine by absorbing the urine into a porous material, (b) identifying when a sufficient volume of sample is collected, and (c) extracting the urine from the porous material.

In embodiments, the porous material is attached to a housing that detachably holds the porous material. In embodiments, the housing that detachably and hingably attached to a rod. In embodiments, the rod is at least 6 inches, or at least 1 foot, or at least 2 feet long, or at least 3 feet long, or at least 4 feet long, or at least 5 feet long.

In embodiments, the porous material is a sponge. In embodiments, the sponge material is cellulose. In embodiments, the porous material is compressed and expands when it absorbs liquid. In embodiments, the porous material changes color when exposed to liquid.

In embodiments, the porous material containing the sample is placed into a sterile air-tight container for transport.

In embodiments, the method further comprises extracting a liquid sample from the porous material.

In embodiments, the liquid sample is extracted from the porous material by compressing the porous material comprising the urine sample. In embodiments, the method further comprises transferring the liquid sample to a sterile air-tight container. In embodiments, the sample is removed from the sponge immediately before analysis at a laboratory.

In embodiments, the method: (a) minimizes the effort required by the collector to obtain the urine sample, and/or (b) minimizes the risk of sample contamination.

In aspects, the present disclosure provides a method for performing urine analysis, the method comprising collecting urine using the urine collection device of any one of the embodiments disclosed herein, or the kit of the embodiments disclosed herein, or the methos of any the embodiments disclosed herein and performing urine analysis.

In embodiments, the analysis is performed at a laboratory. In embodiments, the analysis includes a quantitative protein and creatinine analysis, a urine dip stick analysis (e.g., a colorimetric test for a variety of analytes), a sedimentation analysis, and/or a urine culture identification with sensitivity (e.g., for pathogen identification, for example for a bacterial infection and/or a viral infection).

### Subjects

In embodiments, the subject is a non-human animal. In embodiments, the non-human animal is a captive animal. In embodiments, the non-human animal is a pet animal, a farm animal, or a zoo animal. In embodiments, the subject is a mammal, e.g., dog, cat, horse, cow, pig, rabbit, sheep, mouse, rat, guinea pig, or non-human primate, such as a monkey, chimpanzee, or baboon. In embodiments, the subject is a canine or a feline. In embodiments, the subject is a dog (i.e. a canine), or other members of the family Canidae.

### Kits

In aspects, the present disclosure provides a kit for urine collection comprising the urine collection device of any one of any of the embodiments disclosed herein.

In embodiments, the kit further comprises one or more of a urine collection vial, a container to hold dry and/or wet porous material, one or more sets of porous material (e.g., one or more disposable sponges), a sterile air-tight container for transport, and instructions to use. In embodiments, the kit further comprises shipping materials, including a box and/or shipping label. In embodiments, the kit includes gloves and/or other personal protective equipment for aseptic handling of urine samples. In embodiments, the kit includes a sealable transport container, such as a sealable biohazard bag.

In embodiments, the kit further comprises one or more of positive control samples, negative control samples, a key for estimating a number of viable cells of a microorganism, and instructions to use. In embodiments, the kit further comprises a collection tube.

### EXAMPLES

The examples herein are provided to illustrate advantages and benefits of the present disclosure and to further assist a person of ordinary skill in the art with preparing and using the urine collection device and kits of the present disclosure. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: Selection of Sponge Material

Several sponge materials from different manufacturers were tested. Sponges had different capacities for the amount of liquid they could contain per unit volume, i.e. absorbance efficiency.

Releasing the liquid from the sponge was easier if the sponge was completed saturated with the liquid. The sponge material selected had a relatively low absorbance efficiency.

The size of the dry sponge was tested to determine the sample volume required to extract a minimum of 1 mL of sample from the sponge for analysis.

The following experiment was performed to confirm that the sample can be removed from the porous material after storage and/or transport. The size of the sponge tested was 3.5×5 cm. Different amounts of water were added to sponge samples and the sponges were placed inside of Ziplock bags and sealed. Every 24 hours, a set of sponges was removed from their Ziplock bags and squeezed. The amount of water released from each sponge was measured. The results are shown in Table 1.

TABLE 1

| | Released after | Released after | Released after |
|---|---|---|---|
| | 24 h (mL) | 48 h (mL) | 72 h (mL) |
| 10 mL water | | | |
| added | | | |
| #1 | 4.0 | 2.4 | 2.0 |
| #2 | 4.1 | 2.5 | 2.1 |
| #3 | 3.9 | 3.8 | 1.8 |
| #4 | 2.1 | 4.1 | 3.7 |
| 20 mL water | | | |
| added | | | |
| #1 | 13 | 10 | 14 |
| #2 | 13 | 10 | 14 |
| #3 | 13 | 10 | 13 |
| #4 | 14 | 10 | 12 |

### Example 2: Urinalysis of Urine Sample Collected Using a Sponge of the Present Technology

The following experiment was performed to confirm that the sponge material does not affect the urinalysis or culture test results.

A fresh human urine sample was collected into a sterile collection cup using the free catch technique. 2 mL of the sample was sent for analysis as a control. 20 ml of sample was applied onto each of 2 sponges. The sponges were placed in a plastic bag. The plastic bag was sealed, and the samples were left on the sponge for 15 minutes or 1 hour. The bag was then opened, and the sponge was squeezed so that the sample was expressed from the sponge. A sterile dropper was used to collect 2 mL of sample from each bag. The processed samples were then sent for analysis. The analysis results for the 3 samples are shown in Table 2.

TABLE 2

| | | 15 min on | 60 min on |
|---|---|---|---|
| | Control | sponge | sponge |
| Urine Protein | 8.3 | 11.6 | 10.5 |
| Urine Creatinine | 0.1 | 0.1 | 0.1 |
| UPCR | 136.0 | 132.0 | 134.0 |
| Color | Yellow | Yellow | Yellow |
| Clarity | Cloudy | Cloudy | Cloudy |
| Specific Gravity | 1.016 | 1.017 | 1.016 |
| pH | 6.0 | 6.0 | 6.0 |
| Protein | Negative | Negative | Negative |
| Glucose | Negative | Negative | Negative |
| Ketones | Negative | Negative | Negative |
| Urobilinogen | Normal | Normal | Normal |
| Bilirubin | Negative | Negative | Negative |
| Blood | Negative | Negative | Negative |
| WBC (/HPF) | 0-2 | 0 | 0 |
| RBC (/HPF) | 0-2 | 0 | 0 |
| Bacteria (/HPF) | <8 | <8 | <8 |
| EPI Cells (/HPF) | 0-1 | 0-1 | 0-1 |
| Mucus | None Seen | None Seen | None Seen |
| Casts (/LPF) | None Seen | None Seen | None Seen |
| Crystals (/HPF) | 1 + Calcium Oxalate | None Seen | None Seen |
| | Dihydrate (1-5), | | |
| | Amorphous Urates | | |
| | Present | | |
| Other Notes | Amorphous | Amorphous | Amorphous |
| | Debris | Debris | Debris |

The present sponge may have not released the crystals, or they may have not been observed by the technician performing the analysis as they were rare in the original sample and amorphous debris was noted in all of the samples which may have made the observation difficult. Additional testing will be performed to determine what affect the sponge has on crystal detection in urine samples. Overall, the results demonstrated, inter alia, that the sponge collection mode did not substantially alter the concentrations of the components of the urine samples.

### Example 3: Urinalysis of Sponge Collection Compared Against Cystocentesis

Urine samples were collected from 33 companion animals by cystocentesis, e.g., obtaining urine directly from the urinary bladder by inserting a needle through the body wall. The urine samples were split into 2 aliquots, where one aliquot was applied to the QSM sponge, and the second aliquot was directly placed into a sterile urine transport tube. After 5 minutes, the urine was extracted from the present sponge, by manually expressing the urine from the sponge into a sterile plastic bag. An eye dropper was then used to aliquot the urine into a sterile urine transport tube. Two aliquots for each sample were submitted for analysis. The following tests were performed on all of the submitted samples: quantitative protein and creatinine, urine dip stick, sedimentation analysis, and urine culture identification with sensitivity.

Values that were identical between the two aliquots were reported as a match. Where a discrepancy was measured between the values for a given sample, the magnitude of the discrepancy was binned.

Quantitative values were obtained for urine protein and urine creatinine. For these measurements, each bin represented a deviation of 10 mg/dL. For specific gravity, each bin represented 0.005 units. For pH, each bin represented 1 pH unit. Protein, glucose, ketones, urobilinogen, bilirubin, and blood values were obtained from the dip stick analyses, and were reported as 0, 1+, 2+, 3+, 4+ based on a colorimetric change from each analyte. Microscopic analyses were grouped as Not Seen, 0-2, 10-15, 50-75, or >100 per cell count on a low powered magnification field for white blood cells (WBC), red blood cells (RBC), epithelial (EPI) cells, casts, and crystals. Bacterial cell counts were grouped by cell count as Not seen, Rare (<8), Moderate (9-40), or Marked (>40) per high powered magnification field. Two types of crystals were reported: Ammonium Magnesium Phosphate (Triple Phosphate) and Calcium Oxalate Monohydrate/Dihydrate (CaOx). The results of the analyses are summarized in Table 3 below.

TABLE 3
Comparison of results from urine samples obtained from 33 companion
animals. Samples were collected via cystocentesis. Half of
each sample was deposited directly (Cysto) absorbed into the
present sponge and released after 5 minutes (Sponge).

| | Sponge value higher | | | | Cysto value higher | | |
|---|---|---|---|---|---|---|---|
| | 3+ | 2+ | 1+ | Match | 1− | 2− | 3− |
| Urine Protein | | | | 32 | 1 | | |
| Urine Creatinine | | | | 31 | 2 | | |
| Specific Gravity | | | | 33 | | | |
| pH | | | | 31 | 1 | 1 | |
| Protein | | | | 33 | | | |
| Glucose | | 1 | | 32 | | | |
| Ketones | | | 1 | 32 | | | |
| Urobilinogen | | | 0 | 33 | | 0 | |
| Bilirubin | | | | 32 | 1 | | |
| Blood | | | 1 | 31 | | 1 | |
| WBC | | | 4 | 22 | 7 | | |
| RBC | 1 | | | 23 | 6 | 2 | 1 |
| EPI Cells | | | | 32 | 1 | | |
| Mucus | | | 1 | 30 | 2 | | |
| Casts | | | | 31 | 2 | | |
| Triple Phosphate | | 1 | 3 | 25 | 4 | | |
| Crystals | | | | | | | |
| CaOx Crystals | 1 | 1 | 1 | 30 | | | |
| Bacteria | | 16 | | 16 | 1 | | |
| Culture ID | | | | 33 | | | |

In 16 of the present sponge samples, there was a moderate or marked increase in the number of bacterial cells with microscopic analysis. However, in none of these samples was any bacterial growth detected on culture plates, suggesting that the laboratory most likely observed debris from the sponge and not bacterial cells. Overall, the resulted demonstrated, inter alia, that the porous material did not substantially bind to the tested components of the urine samples, nor did it substantially effect the results of the concentrations of these components. Moreover, the porous material and/or the method handling the urine samples using the porous material did not substantially introduce contaminants into the urine samples that would alter the results of the components.

### EQUIVALENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure come within known or customary practice within the art to which the disclosure pertains and as may be applied to the essential features hereinbefore set forth and as follows in the scope of the appended claims.

### INCORPORATION BY REFERENCE

All patents and publications referenced herein are hereby incorporated by reference in their entireties, including U.S. Provisional Application No. 63/394,080, filed Aug. 1, 2022. The disclosure has been described in an illustrative manner, and it is to be understood that the terminology which has been used is intended to be in the nature of words of description rather than of limitation.

What is claimed is:

1. A urine collection device comprising
(a) a urine collection substrate comprising a porous material,
(b) a housing for the porous material that can detachably hold the porous material; and
(c) a rod that is detachably attachable to the sponge housing.

2. The urine collection device of claim 1, wherein the porous material is a sponge.

3. The urine collection device of claim 2, wherein the sponge is cellulose sponge or hydrophilic polyurethane sponge.

4. The urine collection device of any one of claims 1 to 3, wherein the porous material is expandable upon contact with an aqueous liquid.

5. The urine collection device of any one of claims 1 to 4, wherein the porous material is compressed and expands when it absorbs liquid.

6. The urine collection device of any one of claims 1 to 5, wherein the porous material changes color when exposed to liquid.

7. The urine collection device of any one of claims 1 to 6, wherein the porous material does not substantially bind to one or more components of urine selected from proteins, electrolytes, creatinine, urine pigments (e.g., urochrome, and pigments from food such as beets), acids, bases, ammonia, glucose, ketones, blood cells, epithelial cells, bacteria, mucus, bilirubin, urinary casts, and urinary crystals.

8. The urine collection device of any one of claims 1 to 7, wherein the porous material does not substantially release in urine sample absorbed in the porous material one of more of pigments, sugars, acids, bases, ketones, ammonia, bacteria and electrolytes.

9. The urine collection device of any one of claims 1 to 8, wherein when a urine specimen that is extracted from the porous material that has absorbed a urine sample, the urine specimen does not substantially differ from the urine sample with respect to one or more of pH, specific gravity, and electrolytes.

10. The urine collection device of any one of claims 1 to 7, wherein the sponge has a capacity of absorbing at least 0.5 mL, or at least 1 mL, or at least 2 mL, or at least 5 mL, or at least 10 mL, or at least 20 mL aqueous liquid.

11. The urine collection device of any one of claims 1 to 10, wherein the rod is hingably attachable to the sponge housing.

12. The urine collection device of any one of claims 1 to 11, wherein the rod is foldable and/or extendable.

13. The urine collection device of any one of claims 1 to 12, wherein the urine collection device further comprises a handle.

14. The urine collection device of any one of claims 1 to 13, wherein the urine collection device: (a) holds the porous collection material in place, (b) minimizes the effort required by the collector to obtain the urine sample, and/or (c) minimizes the risk of sample contamination.

15. A kit for urine collection comprising the urine collection device of any one of claims 1 to 14.

16. The kit of claim 15, further comprising one or more of a urine collection vial, a container to hold dry and/or wet porous material, a sterile air-tight container for transport, and instructions to use.

17. A method for collecting urine, the method comprising:
(a) obtaining a sample of urine by absorbing the urine into a porous material,
(b) identifying when a sufficient volume of sample is collected, and
(c) extracting the urine from the porous material.

18. The method of claim 17, wherein the porous material is attached to a housing that detachably holds the porous material.

19. The method of claim 18, wherein the housing that detachably and hingably attached to a rod, the rod is at least 6 inches, or at least 1 foot, or at least 2 feet long.

20. The method of claim any one of claims 17 to 19, wherein the porous material is a sponge.

21. The method of claim 20, wherein the sponge material is cellulose.

22. The method of any one of claims 17 to 21, wherein the porous material is compressed and expands when it absorbs liquid.

23. The method of any one of claims 17 to 22, wherein the porous material changes color when exposed to liquid.

24. The method of any one of claims 17 to 23, wherein the porous material containing the sample is placed into a sterile air-tight container for transport.

25. The method of any one of claims 17 to 24, the method further comprises extracting a liquid sample from the porous material.

26. The method of claim 25, wherein the liquid sample is extracted from the porous material by compressing the porous material comprising the urine sample.

27. The method of claim 25 or claim 26, the method further comprises transferring the liquid sample to a sterile air-tight container.

28. The method of claim 26 or claim 27, wherein the sample is removed from the sponge immediately before analysis at a laboratory.

29. The method of any one of claims 17 to 28, wherein the method: (a) minimizes the effort required by the collector to obtain the urine sample, and/or (b) minimizes the risk of sample contamination.

30. A method for performing urine analysis, the method comprising collecting urine using a urine collection device of any one of claims 1 to 14, or a kit of claim 15 or claim 16.

31. The method of claim 30, wherein the analysis is performed at a laboratory.

32. The method of claim 30 or 31, wherein the analysis comprises a quantitative analysis of urine protein and/or urine creatinine, urine dip stick analysis, urine sedimentation analysis, and/or urine culture identification.
